(19) United States
(12) Patent Application Publication
(10) Pub. No.: US 2008/0243069 A1
(43) Pub. Date: Oct. 2, 2008

(54) Self-Crimping Radiopaque marker

(75) Inventor: Michael Krivoruchko, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/695,468

(22) Filed: Apr. 2, 2007

(51) Int. Cl. A61M 37/00

(52) U.S. Cl. 604/103.1

(57) ABSTRACT

A radiopaque marker band includes a tube having an inner surface and an outer surface. The tube is made from a shape-memory material, such as a nickel-titanium alloy. A coating is disposed on at least a portion of the outer surface of the tube. The coating has a greater radiopacity that said shape-memory material. The coating may be applied in a plurality of bands on the outer surface of the tube.

### FIELD OF THE INVENTION

This invention relates generally to radiopaque markers and, in particular, to a radiopaque marker band made of a shape-memory material coated with a radiopaque material.

### BACKGROUND OF THE INVENTION

Many current procedures for treating a patient include the use of medical instruments that are inserted into the patient's vasculature, such as catheters for use in angioplasty and stenting procedures. In performing intravascular procedures, a physician typically uses a fluoroscope to visualize a patient's vascular structure. It is known to use one or more marker bands affixed to the medical instrument such as a catheter to assist the physician in guiding and positioning the catheter within the patient's vascular system.

Known marker bands are typically constructed of a solid band of radiopaque material, such as platinum, iridium, tungsten, tantalum, gold, etc. and alloys thereof. Typically, the marker band is slipped around and onto a shaft of the catheter and then affixed to the shaft with an adhesive, crimping, or by heating the shaft.

Vascular structures can be very tortuous, and marker bands attached to shafts as described above increase the outer diameter of the shafts. U.S. Pat. No. 5,485,667 describes a marker band made of a shape-memory material such as a nickel-titanium alloy, however, nickel-titanium alloys are not sufficiently radiopaque to be used as suitable marker bands in applications where the marker band must have a small thickness, such as in coronary applications.

### BRIEF SUMMARY OF THE INVENTION

A radiopaque marker band includes a tube having an inner surface and an outer surface. The tube is made from a shape-memory material, such as a nickel-titanium alloy. A radiopaque coating is disposed on an outer surface of the tube, the coating having a grater radiopacity than the shape-memory material. The radiopaque coating may be, for example, platinum, iridium, tungsten, tantalum, gold, or alloys thereof, or any other suitable radiopaque material.

The radiopaque marker band may be attached to a medical instrument by utilizing the shape-memory property of the shape-memory material. The marker band is formed at an original configuration with its outer diameter approximately equal to the outer diameter of a tubular member of the medical instrument. At a first temperature, the marker band is deformed to a deformed configuration in which an inner diameter of the marker band is greater than the outer diameter of the tubular member and the marker band is placed concentrically around the tubular member. The temperature of the shape-memory material of the marker band is increased to a second temperature such that the marker band returns to its original configuration, thereby contracting around the tubular member.

Alternatively, the original configuration of the marker band has an inner diameter that is approximately equal to the inner diameter of the tubular member. At a first temperature, the marker band is deformed to a deformed configuration in which an outer diameter of the marker band is smaller than the inner diameter of the tubular member and the marker band is placed inside the lumen of the tubular member. The temperature of the shape-memory material of the marker band is increased to a second temperature such that the marker band returns to its original configuration, thereby expanding the marker band to secure it to the inner surface of the tubular member.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a side, partial cut-away view of a catheter including radiopaque marker bands.

FIG. 2 illustrates a perspective view of a radiopaque marker band in accordance with an embodiment of the present invention.

FIG. 3 illustrates a cross-sectional view of the radiopaque marker band of FIG. 2.

FIG. 4 illustrates a cross-sectional view of a radiopaque marker band in accordance with another embodiment of the present invention.

FIG. 5 illustrates a perspective view of a radiopaque marker band in accordance with another embodiment of the present invention.

FIG. 6 illustrates a cross-sectional view of a radiopaque marker band in a deformed configuration surrounding a tubular member.

FIG. 7 illustrates a cross-sectional view of a support rod inserted into the tubular member of FIG. 6.

FIG. 8 illustrates a cross-sectional view of the tubular member of FIG. 6 with the radiopaque marker band in its original configuration attached to the tubular member.

FIG. 9 illustrates a cross-sectional view of the tubular member of FIG. 8 with the support rod removed from the tubular member.

FIG. 10 illustrates a cross-sectional view an alternative embodiment of a radiopaque marker band in a deformed configuration within a lumen of a tubular member.

FIG. 11 illustrates a cross-sectional view of an alternative the embodiment of FIG. 10 with a support tube surrounding the tubular member.

FIG. 12 illustrates a cross-sectional view of the tubular member of FIG. 10 with the radiopaque marker band in its original configuration attached to the tubular member.

FIG. 13 illustrates a cross-sectional view of the tubular member of FIG. 11 with the tubular member removed from the support tube.

### DETAILED DESCRIPTION OF THE INVENTION

Specific embodiments of the present invention are now described with reference to the figures, where like reference numbers indicate identical or functionally similar elements.

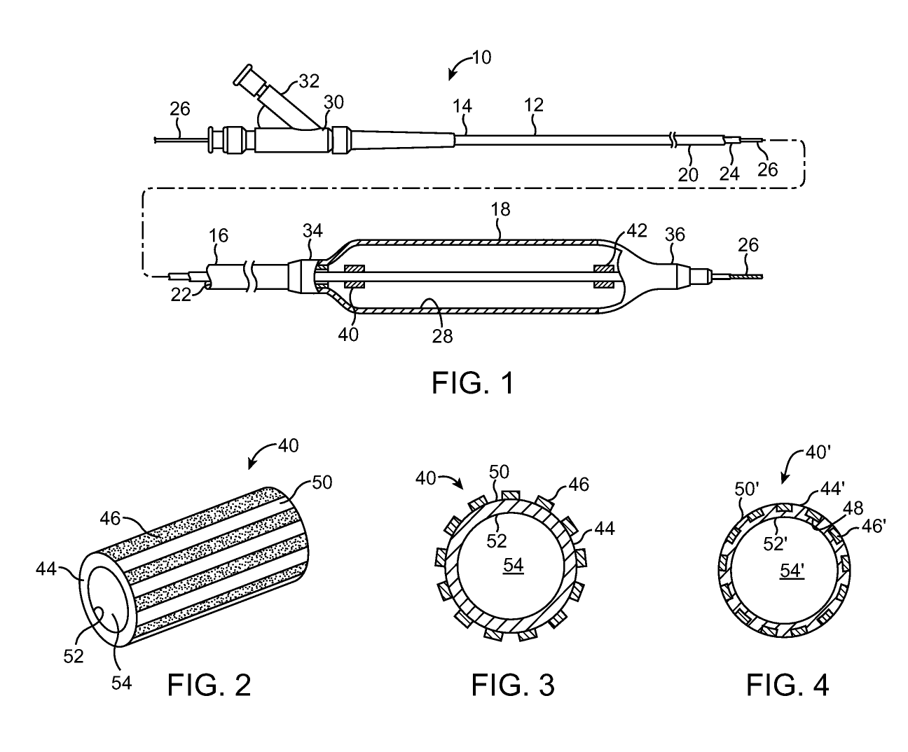

FIG. 1 illustrates an embodiment of an intraluminal catheter 10 of the invention, generally comprising an elongated shaft 12 having a proximal end 14 and a distal end 16, and a balloon 18 on a distal shaft section. In the embodiment illustrated in FIG. 1, the shaft 12 comprises an outer tubular member 20 defining an inflation lumen 22, and an inner tubular member 24 disposed within the outer tubular member and defining a guidewire lumen 25 configured to slidably receive a guidewire 26. In the illustrated embodiment, the coaxial relationship between outer tubular member 20 and inner tubular member 24 defines annular inflation lumen 22. A proximal portion 34 of balloon 18 is sealingly secured to a distal portion of outer tubular member 20 and a distal portion 36 of balloon 18 is sealingly secured to a distal portion of inner tubular member 24, so that an interior 28 of balloon 18 is in fluid communication with inflation lumen 22. An adapter 30 at the proximal end of the shaft 12 is configured to direct inflation fluid through arm 32 into inflation lumen 22, and provide access to the guidewire lumen. Guidewire 26 is disposed within the guidewire lumen.

When using catheter 10 as described with respect to FIG. 1, it is desirable to know the proximal and distal limits of the inflatable portion of balloon 18. This is the working area of balloon 18 when performing a procedure such as PTCA. Also, if delivering an endoprosthesis, such as a stent, mounted on balloon 18, the proximal and distal ends of such an endoprosthesis generally align with the proximal and distal limits of inflatable portion of balloon 18. Materials that are used for catheter 12 and balloon 18 are generally radiolucent such that the materials cannot be viewed by a physician via radiography or fluoroscopy. Thus, in order for a physician to place catheter 12 in the appropriate location with respect to a procedure being performed, radiopaque marker bands 40 and 42 are provided on inner tubular member 24, aligned with the proximal and distal limits of the inflatable portion of balloon 18, as shown in FIG. 1. It would be appreciated by those of ordinary skill in the relevant art that radiopaque marker bands 40, 42 may be placed at other locations along catheter 12. For example, a radiopaque marker band may be placed on inner tubular member 24 at the center point between the proximal and distal limits of the inflatable portion of balloon 18. Further, marker bands may be placed at locations along outer tubular member 20, if desired.

As shown in FIG. 2, radiopaque marker band 40 is a tubular body 44 having an outer surface 50 and an inner surface 52 defining a center bore 54. Tubular body 44 is formed of a shape-memory material, for example, a nickel titanium alloy generally referred to by the acronym “nitinol”. A shape memory material such as nitinol includes a Martensitic (low temperature) phase and an Austenitic (higher temperature phase). In the Martensitic phase, the material may be deformed to a new shape and will maintain that shape. However, upon heating above the Austenite Finish (Af) temperature, the deformation is lost and the material will return to its pre-deformed, original shape. Only suitable shape memory materials, include Cu—Zn or Cu—Al; Cu—Zn—Al; and Cu—Al—Ni; Au—Pt among others. Tubular body 44 may have a thickness defined between inner surface 52 and outer surface 50 of less than or equal to 0.001 inch.

Because shape-memory materials such as nitinol are not sufficiently radiopaque to be used effectively as a radiopaque marker in certain applications, a coating 46 of radiopaque material, such as platinum, iridium, tungsten, tantalum, gold, or alloys thereof, or any other suitable radiopaque material, is disposed in bands on outer surface 50 of tubular body 44. Radiopaque material 46 has a greater radiopacity than the shape-memory material of tubular body 44. Radiopaque material 46 maybe deposited on outer surface 50 by sputtering, plasma deposition, reactive sputtering, physical vapor deposition, chemical vapor deposition, cathodic arc vacuum deposition, electrodeposition, or other deposition techniques, as would be understood by those of ordinary skill in the art. Although FIGS. 2 and 3 show coating 46 deposited in equal bands along the outer surface 50 of tubular body 44, one of ordinary skill in the art would understand that coating 46 can be distributed in any pattern on outer surface 50. Coating 46 may be applied with a thickness in the range of 5 to 25 μm.

FIG. 4 shows an alternative embodiment of a radiopaque marker band 40′ that is a tubular body 44′ having an outer surface 50′ and an inner surface 52′ defining a center bore 54′. As in the embodiment described with respect to FIG. 2, tubular body 44′ is formed of a shape-memory material, for example, a nickel-titanium alloy generally referred to by the acronym “nitinol”. In the embodiment of FIG. 4, slots 48 are provided on outside radial surface 50′. A coating 46′ of radiopaque material, such as platinum, iridium, tungsten, tantalum, gold, or alloys thereof, or any other suitable radiopaque material, is deposited in slots 48. Radiopaque coating 46′ maybe deposited in slots 48 by sputtering, plasma deposition, reactive sputtering, physical vapor deposition, chemical vapor deposition, cathodic arc vacuum deposition, electrodeposition, or other deposition techniques, as would be understood by those of ordinary skill in the art. By applying radiopaque coating 46′ in slots 48, the overall thickness of radiopaque marker band 40′ is reduced.

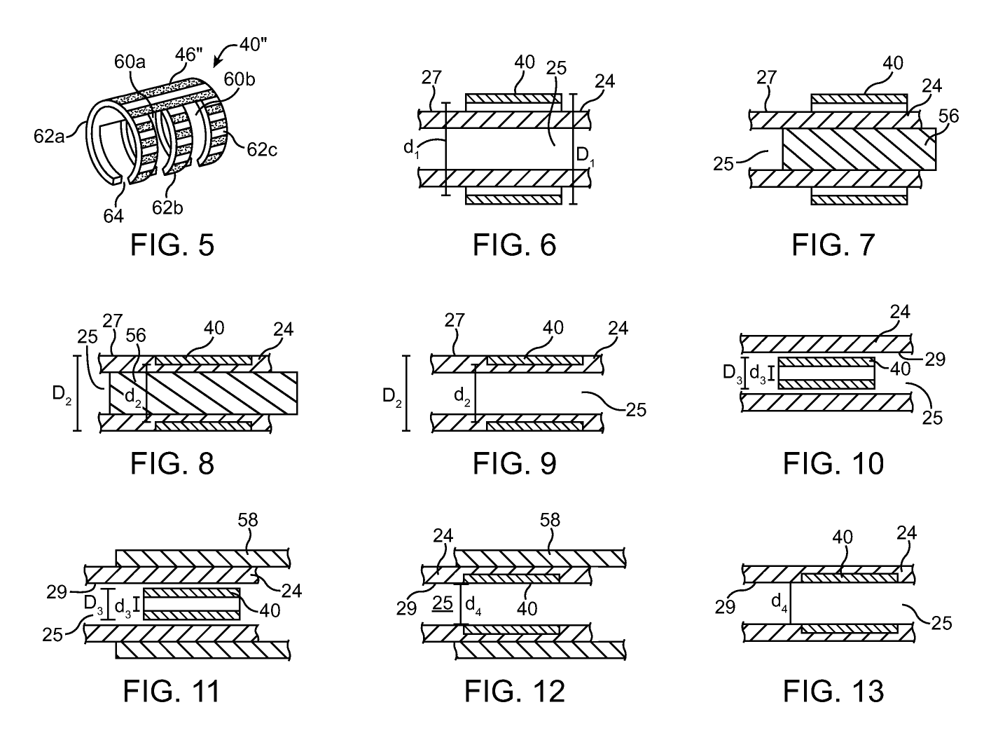

FIG. 5 shows an alternative embodiment of a radiopaque, self-crimping marker band 40″. Marker band 40″ includes two notches 60a, 60b and three segments 62a, 62b, 62c. Additionally, radiopaque marker band 40″ may include a longitudinal slit 64 running from one end of the marker band to the other end of the marker band. Notches 60a, 60b may be formed using a variety of techniques known in the art, such as laser cutting, as would be known to one of ordinary skill in the art. Marker band 40″ is made of a shape-memory material such as nitinol and includes a coating 46″ of radiopaque material, such as platinum, iridium, tungsten, tantalum, gold, or alloys thereof, or any other suitable radiopaque material, disposed in bands on outer surface 50″ of tubular body 44″. Marker band 40″ including notches 60a, 60b is more flexible than a similar marker band without notches 60a, 60b. One of skill in the art will recognize that marker band 40″ may have as many notches and thus have as many segments to achieve the desired flexibility.

Radiopaque marker band 40, 40′, 40″ is attached to inner tubular member 24 by the thermally induced recovery of the shape-memory alloy from a deformed configuration as to a pre-deformed, original shape. Marker band 40 in its pre-deformed, original shape has an outer diameter D₂ and an inner diameter d₂, wherein the outer diameter D₂ is substantially equal to or less than the outer diameter of inner tubular member 24, as shown in FIGS. 8 and 9. Marker band 40 is then cooled to a temperature below the shape recovering transition temperature (A_{f}) of the shape-memory material of marker band 40 so as to cause the shape-memory material to become capable of physical deformation by an outside force. Cooling of marker band 40 may be accomplished by any conventional cooling device capable of lowering the temperature of the shape-memory material into the desired low temperature range, such as a cooling device using liquid nitrogen.

While marker band 40 is at the low temperature, it may be deformed into a deformed configuration shown in FIGS. 6 and 7 wherein the inner diameter d₁ of marker band 40 is larger than the outer diameter of inner tubular member 24. Marker band 40 may be deformed to the larger diameter by applying a radially outward force to inner surface 52 of marker band 40 by, for example, forcing a shaping rod through center bore 54 of marker band 40. The shaping rod should have an outer diameter no less than the outer diameter of inner tubular member 24 so that inner diameter d₁ of marker band 40 in the deformed condition may be slid over inner tubular member 24. With marker band 40 in its deformed condition, marker band 40 is positioned concentrically around inner tubular member 24, as shown in FIG. 5.

Prior to raising the temperature of the shape-memory material of marker band 40, a supporting mandrel or rod 56 may be inserted through inner lumen 25 of inner tubular member 24 so as to extend longitudinally within inner tubular member along the length corresponding to the location where marker band 40 is positioned outside of inner tubular member 24, as shown in FIG. 7. The temperature of the shape-memory material of marker band 40 is then raised above the predetermined transition temperature of the shape-memory material (A_{f}) by heating marker band 40 using, for example, hot air or induction-type heating. As the shape-memory material of marker band 40 is raised to a temperature above the transition temperature, marker band 40 begins to return to its smaller diameter original configuration by moving radially inward into contact with outer radial surface 27 of inner tubular member 24. Continued contraction of marker band 40 to its original shape causes inner surface 52 of marker band 40 to press against outer surface 27 of inner tubular member 24. The temperature of marker band 40 may be high enough to soften the material of inner tubular member 24 immediately adjacent to marker band 40. As a result, marker band 40 may sink into the material of inner tubular member 24 until it reaches its original configuration, as shown in FIGS. 8 and 9. During the thermally induced deformation or shape recovery process into the original configuration, support rod 56 supports inner surface 29 of inner tubular member 24 against the radially inward force of marker band 40, thereby maintaining the inside diameter of inner tubular member 24. Support rod 56 is then removed resulting in a radiopaque marker band 40 securely embedded in inner tubular member 24, as shown in FIG. 9.

Referring to FIGS. 10-13, a second embodiment of the present invention is shown which is the same as the first embodiment except that radiopaque marker band 40, 40′, 40″ is embedded into inner surface 29 of inner tubular member 24. Marker band 40 is made of a shape-memory material capable of changing shape from a deformed configuration shown in FIGS. 10 and 11 to an original configuration shown in FIGS. 12 and 13. Marker band 40 includes a coating 46 of radiopaque material, as described above with respect to FIGS. 2-5. Marker band 40 is sized, or chosen, so that it has an inner diameter d₄ when in its original configuration which is no smaller than, and preferably approximately equal to, the inner diameter of lumen 25 formed by inner surface 29 of inner tubular member 24. After being cooled below the shape recovering transition temperature (A_{f}) of the shape-memory material of marker band 40 so as to cause the shape-memory material to become capable of physical deformation by an outside force, marker band 40 is deformed into the configuration shown in FIGS. 10 and 11. Marker band 40 is deformed such that an outer diameter D₃ of marker band 40 in the deformed condition is less than the diameter of lumen 25 bounded by inner surface 29 of inner tubular member 24. Marker band 40 may then be slid into lumen 25 of inner tubular member 24, as shown in FIG. 10. Inner tubular member 24 may then be slid into a bore of a support tube 58 having an inner diameter approximately equal to the outer diameter of inner tubular member 24, as shown in FIG. 11. The temperature of the shape-memory material of marker band 40 is then raised above the predetermined transition temperature of the shape-memory material (A_{f}) by heating marker band 40. This causes marker band 40 to begin to return to its larger diameter original configuration by moving radially outward into contact with inner surface 29 of inner tubular member 24. Continued expansion of marker band 40 to its original shape causes outer surface 50 of marker band 40 to press against inner surface 29 of inner tubular member 24. The temperature of marker band 40 may be high enough to soften the material of inner tubular member 24 immediately adjacent to marker band 40. As a result, marker band 40 may sink into the material of inner tubular member 24 until it reaches its original configuration, as shown in FIGS. 12 and 13. During the thermally induced deformation or shape recovery process into the original configuration, support tube 58 supports outer surface 27 of inner tubular member 24 against the radially outward force of marker band 40, thereby maintaining the outside diameter of inner tubular member 24. Support tube 58 is then removed resulting in a radiopaque marker band 40 securely embedded in inner tubular member 24, as shown in FIG. 13.

Although marker bands 40, 40′, 40″ have been described as being used with respect to inner tubular member 24 of the embodiment of FIG. 1, one of ordinary skill in the art would recognize that marker bands 40, 40′, 40″ may be used in a variety of applications. For example, and not by way of limitation, marker bands 40, 40′, 40″ may be attached to outer shaft 20, may be used to attach a balloon or other device to a shaft, or may be used in other applications wherein it is necessary to provide a radiopaque marker band. Further, although the marker bands have been illustrated as completely embedding into either the outer surface or inner surface of inner tubular member 24, one of ordinary skill in the art would recognize that complete embedment may not be necessary depending on the application. Thus, marker band 40, 40′, 40″ may partially embed into outer or inner surface of the shaft to which it is being attached or may simply constrict (or expand if attached to an inner surface) sufficiently so as to provide sufficient frictional force between the marker band and the shaft such that the marker band will not slide along the shaft.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A radiopaque marker band comprising:
a tube having an inner surface and an outer surface, wherein the tube is made from a shape-memory material; and
a coating disposed on at least a portion of the outer surface, wherein the coating has a greater radiopacity than the shape-memory material.

2. The radiopaque marker band of claim 1, wherein the shape-memory material is a nickel-titanium alloy.

3. The radiopaque marker band of claim 1, wherein the coating comprises a plurality of bands.

4. The radiopaque marker band of claim 1, wherein the coating is deposited on the tube by one of sputtering, plasma deposition, reactive sputtering, physical vapor deposition, chemical vapor deposition, cathodic arc vacuum deposition, or electrodeposition.

5. The radiopaque marker band of claim 1, wherein the coating is selected from the group consisting of platinum, iridium, tungsten, tantalum, gold, and alloys thereof.

6. The radiopaque marker band of claim 1, wherein the coating has a thickness in the range of 5 to 25 μm.

7. The radiopaque marker band of claim 1, further comprising slots in the outer surface of the tube, wherein the coating is disposed in the slots.

8. The radiopaque marker band of claim 1, wherein the tube includes circumferential notches creating segments in the tube.

9. The radiopaque marker band of claim 1, wherein the tube has a thickness of 0.001 inch or less.

10. An intraluminal device comprising:
a first tubular member;
a second tubular member coupled to the first tubular member, wherein the second tubular member includes an outer surface and wherein the second tubular member comprises a shape-memory material; and
a coating disposed on at least a portion of the outer surface of the second tubular member, wherein the coating has a greater radiopacity than the shape-memory material.

11. The intraluminal device of claim 10, wherein the shape-memory material is a nickel-titanium alloy.

12. The intraluminal device of claim 10, wherein the coating comprises a plurality of bands.

13. The intraluminal device of claim 10, wherein the coating is deposited on the second tubular member by one of sputtering, plasma deposition, reactive sputtering, physical vapor deposition, chemical vapor deposition, cathodic arc vacuum deposition, or electrodeposition, or other deposition techniques.

14. The intraluminal device of claim 10, wherein the coating is selected from the group consisting of platinum, iridium, tungsten, tantalum, gold, and alloys thereof.

15. The intraluminal device of claim 10, wherein the coating has a thickness in the range of 5 to 25 μm.

16. The intraluminal device of claim 10, further comprising slots in the outer surface of the second tubular member, wherein the coating is disposed in the slots.

17. The intraluminal device of claim 10, wherein the second tubular member has a thickness of 0.001 inch or less.

18. The intraluminal device of claim 10, wherein the device is a catheter.

19. The intraluminal device of claim 18, wherein the first tubular member is an inner tubular member of the catheter.

20. A method for attaching a radiopaque marker to a medical instrument used for treating a patient, comprising the steps of:
providing a medical instrument including a tubular member having an inner surface and an outer surface;
providing a marker formed of a shape-memory material capable of having a deformed configuration while at a first temperature and an original configuration at a second, higher temperature, wherein the marker includes a coating disposed on an outer surface of the marker, wherein the coating has a greater radiopacity than the shape memory-material;
cooling the marker to the first temperature;
deforming the marker into the deformed configuration from the original configuration while the shape-memory material is at the first temperature, wherein the deformed configuration has a larger diameter than the original configuration, and the larger diameter is sufficient such that the marker can surround the tubular member;
positioning the deformed marker around the tubular member of the medical instrument;
positioning a support member adjacent the inner surface of the tubular member for supporting the tubular member prior to engaging the marker with the tubular member; and
changing the temperature of the shape-memory material from the first temperature to the second temperature to cause the marker to transform from the deformed configuration to the original configuration, thereby engaging the tubular member.

21. The method of claim 20, wherein the shape-memory material is a nickel-titanium alloy.

22. The method of claim 20, wherein the wherein the coating is selected from the group consisting of platinum, iridium, tungsten, tantalum, gold, and alloys thereof.

23. The method of claim 20, wherein the coating comprises a plurality of bands.

24. The method of claim 20, wherein the marker has a thickness of 0.001 inch or less.

25. A method for attaching a radiopaque marker to a medical instrument used for treating a patient, comprising the steps of:
providing a medical instrument including a tubular member having an inner surface and an outer surface;
providing a marker formed of a shape-memory material capable of having a deformed configuration while at a first temperature and an original configuration at a second, higher temperature, wherein the marker includes a coating disposed on an outer surface of the marker, wherein the coating has a greater radiopacity than the shape-memory material;
cooling the shape-memory material to the first temperature;
deforming the marker into the deformed configuration from the original configuration while the marker is at the first temperature, wherein the deformed configuration has a smaller diameter than the original configuration, and the smaller diameter is sufficient such that the marker can fit within a lumen of the tubular member;
positioning the deformed marker within the lumen of the tubular member of the medical instrument;
positioning a support member adjacent the outer surface of the tubular member for supporting the tubular member prior to engaging the marker with the tubular member; and
changing the temperature of the shape-memory material from the first temperature to the second temperature to cause the marker to transform from the deformed configuration to the original configuration, thereby engaging the tubular member.

26. The method of claim 25, wherein the shape-memory material is a nickel-titanium alloy.

27. The method of claim 25, wherein the wherein the coating is selected from the group consisting of platinum, iridium, tungsten, tantalum, gold, and alloys thereof.

28. The method of claim 25, wherein the coating comprises a plurality of bands.

29. The method of claim 25, wherein the marker has a thickness of 0.001 inch or less.

30. A method of forming a radiopaque marker band comprising the steps of:
providing a tube formed of a shape-memory material that is capable of having a deformed configuration while at a first temperature and an original configuration at a second temperature; and
coating an outside surface of the tube, wherein the coating has a greater radiopacity than the shape-memory material.

31. The method of claim 30, wherein the shape-memory material is a nickel-titanium alloy.

32. The method of claim 30, wherein the coating is applied in a plurality of bands.

33. The method of claim 30, wherein the coating step is selected from the group consisting of sputtering, plasma deposition, reactive sputtering, physical vapor deposition, chemical vapor deposition, cathodic arc vacuum deposition, or electrodeposition.

34. The method of claim 30, wherein the coating is selected from the group consisting of platinum, iridium, tungsten, tantalum, gold, and alloys thereof.

35. The method of claim 30, wherein the coating is applied with a thickness in the range of 5 to 25 μm.

36. The method of claim 30, further comprising the step of providing slots in the outer surface of the tube prior to the coating step, wherein the coating step applies the coating within the slots.

37. The method of claim 30, wherein the tube has a thickness of 0.001 inch or less.
